SEQUENCE LISTING (12 sequences)

SEQ ID NO: 1 - protein, 247 aa, artificial sequence
  huHMFG1-scFv/Clone 4.9
QVQLVQSGAEVKKPGASVKVSCKASGYTFSAYWIEWVRQAPGKGLEWVGEILPGSNNSRYNEKFKGRVTVTADTSTNTAYMELSSLRSEDTAVYYCARSYDFAWFAYWGQGTLVTVSSGGGGSGGGGSGGGGSDIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKR

SEQ ID NO: 2 - DNA, 741 bp, artificial sequence
  huHMFG1-scFv/Clone 4.9
caggtgcagctggtgcagtctggggcagaggtgaaaaagcctggggcctcagtgaaggtgtcctgcaaggcttctggctacaccttcagtgcctactggatagagtgggtgcgccaggctccaggaaagggcctcgagtgggtcggagagattttacctggaagtaataattctagatacaatgagaagttcaagggccgagtgacagtcactgcagacacatccacaaacacagcctacatggagctcagcagcctgaggtctgaggacacagccgtctattactgtgcaagatcctacgactttgcctggtttgcttactggggccaagggactctggtcaccgtctcctcaggtggaggcggttcaggcggaggtggctctggcggtggcggatcggacatccagatgacccagagcccaagcagtctgagcgccagcgtgggtgacagagtgaccatcacctgtaagtccagtcagagccttttatatagtagcaatcaaaagatctacttggcctggtaccagcagaagccaggtaaggctccaaagctgctgatctactgggcatccactagggaatctggtgtgccaagcagattcagcggtagcggtagcggtaccgacttcaccttcaccatcagcagcctccagccagaggacatcgccacctactactgccagcaatattatagatatcctcggacgttcggccaagggaccaaggtggaaatcaaacga

SEQ ID NO: 3 - DNA, 27 bp, artificial sequence
  huHMFG1VL forward primer
catgccatggccgacatccagatgacc

SEQ ID NO: 4 - DNA, 26 bp, artificial sequence
  huHMFG1VL reverse primer
ccgctcgaggctcgtttgatttccac

SEQ ID NO: 5 - DNA, 23 bp, artificial sequence
  huHMFG1VH forward primer
gtagtgcagaggtgcagctggtg

SEQ ID NO: 6 - DNA, 32 bp, artificial sequence
  huHMFG1VH reverse primer
ttagcggccgcggatcctgaggagactgtgac

SEQ ID NO: 7 - protein, 5 aa, artificial sequence
  protein linker sequence
GGGGS

SEQ ID NO: 8 - protein, 17 aa, artificial sequence
  protein linker sequence
ASSGGGGSGGGGSGGSA

SEQ ID NO: 9 - protein, 22 aa, artificial sequence
  protein linker sequence
ASSGGGGSGGGGSGGSAGGGGS

SEQ ID NO: 10 - protein, 15 aa, artificial sequence
  protein linker sequence
GGGGSGGGGSGGGGS

SEQ ID NO: 11 - protein, 13 aa, artificial sequence
  fragment of staphylococcal protein A
AKKLNDAQAPKSD

SEQ ID NO: 12 - DNA, 741 bp, artificial sequence
  complement of SEQ ID NO2
gtccacgtcgaccacgtcagaccccgtctccactttttcggaccccggagtcacttccacaggacgttccgaagaccgatgtggaagtcacggatgacctatctcacccacgcggtccgaggtcctttcccggagctcacccagcctctctaaaatggaccttcattattaagatctatgttactcttcaagttcccggctcactgtcagtgacgtctgtgtaggtgtttgtgtcggatgtacctcgagtcgtcggactccagactcctgtgtcggcagataatgacacgttctaggatgctgaaacggaccaaacgaatgaccccggttccctgagaccagtggcagaggagtccacctccgccaagtccgcctccaccgagaccgccaccgcctagcctgtaggtctactgggtctcgggttcgtcagactcgcggtcgcacccactgtctcactggtagtggacattcaggtcagtctcggaaaatatatcatcgttagttttctagatgaaccggaccatggtcgtcttcggtccattccgaggtttcgacgactagatgacccgtaggtgatcccttagaccacacggttcgtctaagtcgccatcgccatcgccatggctgaagtggaagtggtagtcgtcggaggtcggtctcctgtagcggtggatgatgacggtcgttataatatctataggagcctgcaagccggttccctggttccacctttagtttgct

(19) United States
(12) Patent Application Publication
(10) Pub. No.: US 2006/0193849 A1
(43) Pub. Date: Aug. 31, 2006

(54) Biological materials and uses thereof

(75) Inventors: Juergen Krauss, Essen (DE); Nigel S. Courtenay-Luck, Enfield (GB); Susanna M. Rybak, Green Cove Springs, FL (US)

Correspondence Address: PATREA L. PABST;PABST PATENT GROUP LLP, 400 COLONY SQUARE, SUITE 1200, ATLANTA, GA, 30361, US

(73) Assignees: Antisoma Plc; National Institutes of Health

(21) Appl. No.: 11/066,045

(22) Filed: Feb. 25, 2005

(51) Int. Cl. A61K 39/395; C07K 16/44

(52) U.S. Cl. 424/133.1; 530/387.3

(57) ABSTRACT

A modified antibody molecule which selectively binds to a specific target, the antibody molecule being modified at at least one amino acid residue that determines antigen binding selectivity and/or affinity, characterised in that the modified antibody molecule exhibits a greater stability than an unmodified parent antibody molecule which selectively binds to the target. Also described are nucleotide sequences encoding, vectors, host cells and composition containing and uses of such antibodies, antibody fragments or antibody derivatives.

### FIELD OF THE INVENTION

The present invention relates to antibodies, antibody fragments and antibody derivatives possessing high stability.

### BACKGROUND OF THE INVENTION

Many clinical applications, such as radioimmunoimaging, radioimmunotherapy, or administration of recombinant cytotoxic fusion proteins, favourably employ antibody fragments or small antigen binding molecules such as single chain Fv (scFv) antibodies or multivalent derivatives. These smaller antibody fragments or antigen binding molecules possess advantages over the use of whole antibodies (wild type or humanised) in the IgG format. For example, in contrast to whole immunoglobulins, scFv fragments are capable of penetrating solid tumour tissue efficiently (Yokota, T., et al. (1992) Cancer Res 52:3402) and are rapidly cleared from the circulation (Milenic, D. E. et al. (1991) Cancer Res 51:6363).

It is of paramount importance in clinical applications that a scFv fragment exhibits sufficient affinity to the target antigen while possessing a high degree of stability and a sufficiently long half-life to allow the scFv to reach its target and remain active for a clinically acceptable period. Failure to meet these major requirements can result in insufficient enrichment of scFv molecules in xenografted solid tumours in immunodeficient mice, as shown in Adams, G. P. et al. (1998) Cancer Res 58:485 and Willuda, J. et al. (1999) Cancer Res 59:5758, thus hampering future clinical applications.

Previously known scFv's have exhibited too poor a level of stability and too short a half-life to be of wide clinical use. Therefore, the present invention seeks to solve this problem by providing a stable scFv molecule with a sufficiently long half-life to warrant its clinical use.

The MUC-1 gene product, the membrane mucin glycoprotein (polymorphic epithelial mucin or PEM) has been shown to be over-expressed in most adenocarcinomas (Taylor-Papadimitriou et al. (1999) Biochim Biophys Acta 1455:301). MUC-1 over-expression has been widely associated with poor prognosis in patients with colorectal and gastric carcinoma (Baldus, S. E., et al. (2002) Histopathology 40:440; Utsunomiya, T. et al. (1998) Clin Cancer Res 4:2605). MUC-1 has been found more recently to be over-expressed in a variety of haematological malignancies including acute myelogous leukaemia, chronic lymphocytic leukaemia, and multiple myeloma (Brossart, P. et al.(2001) Cancer Res 61:6846).

The glycosylation of MUC-1 glycoprotein in cancer cells is distinct from expressed mucin in healthy tissue (Hanisch, F. G., and Muller, S. (2000) Glycobiology 10:439). As such, tumour-associated mucin glycoproteins have been identified as representing a valuable target for diagnostic and therapeutic approaches using monoclonal antibodies (mAbs).

Several mAbs have been raised against the highly conserved immunogenic MUC-1 core region possessing tandem repeats of 20 amino acids in the extracellular portion of the MUC-1 glycoprotein (Gendler, S. et al. (1998) J Biol Chem 263:12820). These mAbs include HMFG1 which recognises a MUC-1 epitope with high selectivity (Taylor-Papadimitriou, J. et al. (1981) Int J Cancer 28:17).

HMFG1 is internalised by the cell after it has bound its target antigen (Aboud-Pirak, E. et al. (1988) Cancer Res 48:3188). Therefore, HMFG1 provides a valuable tool for the selective delivery of cytotoxic agents into tumour cells. Consequently, a ⁹⁰Y-murineHMFG1 radioimmunoconjugate was employed in a phase I-II clinical trial in patients with advanced ovarian cancer in an adjuvant setting. Intraperitoneal administration of a single dose of the reagent resulted in a >10 year long term survival of 78% of these patients (Epenetos, A. A. et al. (2000) Int J Gynecol Cancer 10:44).

However, murine HMFG1 is immunogenic in humans. Hence, a humanised version, designated huHMFG1, was generated by grafting the murine antigen binding site onto human frameworks (Verhoeyen, M. E. et al. (1993) Immunology 78:364). huHMFG1 was shown to retain the antigen affinity and same selectivity as the rodent ancestor.

To exploit the potential advantages of antibody fragments, huHMFG1 was reformatted into an scFv fragment. Although this construct exhibited appropriate antigen binding it possesses an extremely short half-life of only <2 hrs when incubated in human serum at 37° C.

The variable heavy (V_{H}) and variable light (V_{L}) domains of the antibody are involved in antigen recognition; a fact first recognised by early protease digestion experiments.

That antigenic selectivity is conferred by variable domains and is independent of the constant domains is known from experiments involving the bacterial expression of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the V_{H} and V_{L} partner domains are linked via a flexible oligopeptide (Bird et al. (1988) Science 242, 423; Huston et al. (1988) Proc. Natl. Acad. Sci. USA 85, 5879) and single domain antibodies (dAbs) comprising isolated V domains (Ward et al. (1989) Nature 341, 544). A general review of the techniques involved in the synthesis of antibody fragments which retain their selective binding sites is to be found in Winter & Milstein (1991) Nature 349, 293-299.

Stability of antibodies and fragments thereof is conferred as either inter-domain stability or intra-domain stability. Examples describing the importance of conserved V_{H}-V_{L} interface residues on (sc)Fv inter-domain stability include Chothia et al., 1985, JMB 186, 651-663 and Tan et al., Biophysical J 75, 1998, 1473-1482 or Chowdhury et al., JMB 281, 1998, 917-928

Examples describing residues being important for providing intra-domain stability include Steipe et al., 1994, JMB 240, 188-192; Wörn and Plückthun, 1998, Biochemistry 37, 13120-13127; Ewert et al., 2003, Biochemistry 42, 1517-1528.

On the basis of solved crystal structures several “key residues” of antibody variable domains which are important for maintaining the main chain conformations of the antigen binding loops have been described including site V_{H}-71 for the production of the CDR-H2 loop main chain conformation (Chothia, C. et al. (1989) Nature 342:877; Tramontano, A. et al. (1990) J Mol Biol 215:175). The CDR main chain conformation of an antibody is well known to be essential to the structure of the antigen binding site and thus the particular target selectivity and affinity for that target of the antibody.

The buried “common core” of immunoglobulin variable domains represents a highly conserved structure of Ig variable domains (when superimposing the C alpha atom distances of common core residues from different antibodies with known crystal structures there is no or extremely little deviation) and about 70% of all residues of each variable domain are associated with the core. Chothia et al. (1998) J. Mol. Biol. 278, pp 457-479 describes in detail residues associated with the common core and their identification. This core is found in different antibodies and would influence the antibody in the same manner as shown in huHMFG-1 in the examples. The six amino acid residues found at the two corners of the inner faces of the β-sheets (V_{L}39/V_{H}40, V_{L}42/V_{H}43, V_{L}45/V_{H}46, V_{L}66/V_{H}71, V_{L}68N_{H}73, V_{L}69/V_{H}76) are identifiable as being hydrophilic or neutral amino acid residues by the methods of Chothia et al. (1998) (above). V_{H}71 being one site of this group exhibits a fairly low degree of conservation.

V_{H}71 is a member of a set of sites which form the so-called “Vernier” zone (Foote, J., and Winter G. (1992) J Mol Biol 224:487) comprising a layer of framework residues which support antigen binding loop conformations and their relative dispositions. The “Vernier” zone has been suggested to play an important role for fine-tuning the selectivity of an antibody for binding to an antigen (Foote, J., and Winter G. (1992) J Mol Biol 224:487).

Amino acid residues with small side chains at position V_{H}71 such as alanine are known to provide a cavity for orienting the CDR-H2 loop in closer proximity to CDR-H1 (Tramontano, A. et al. (1990) J Mol Biol 215:175, Xiang, J. et al. (1995) J Mol Biol 253: 385).

Disruption of the cavity, for example by replacing a small side chain amino acid with a bulkier side chain residue, e.g. arginine was shown, for example, to separate the antigen binding loops CDR-H1 and CDRH2 from each other and expose CDR-H2 loop residue V_{H}52a to the surface (Tramontano, A. et al. (1990) J Mol Biol 215:175) resulting in impaired binding of a chimeric antibody for binding to the TAG72 antigen e.g. Xiang, J. et al. (1995) (J Mol Biol 253: 385), a humanised anti-c-erbB-2 antibody for binding to breast cancer cells (Carter, P., et al. (1992) Proc Natl Acad Sci USA 89:4285) or even a complete loss of antigen binding of a humanised anti-alpha 4 integrin antibody (Leger, 0. J. et al. (1997) Hum Antibodies 8:3).

Although these results underline and confirm the important role Of V_{H}71 in the antigen selective binding of an antibody, the present invention provides the surprising improvement on stability of an antibody or antibody fragment, by modification of the V_{H}71 site compared to antibody molecules without the modification.

The association of stability improvement with the V_{H}71 site was surprising since the location of the site and its known structural role in the antigen binding site would suggest that this residue be of no obvious importance in providing stability to modified antibody molecules.

### BRIEF SUMMARY OF THE INVENTION

The present invention has shown that modification of V_{H}71 can surprisingly influence the stability of the whole scFv molecule with only a minor impact on the affinity of the molecule.

The modification Of V_{H}71 as described in the examples causes the CDRs to be located in closer proximity to each other, thereby increasing the buried surface area between motifs CDR-H1 and CDR-H2 which results in a dramatic increase of the intrinsic V_{H} domain stability of the humanised scFv fragment. Therefore, amino acids involved in antigen specificity and/or affinity determination (e.g. framework residue V_{H}71) play a previously unknown role in providing stability to modified antibody molecules.

In a first aspect of the invention, there is provided a modified antibody molecule which selectively binds to a specific target, the antibody molecule being modified at at least one amino acid residue antigen binding selectivity and/or affinity, characterised in that the modified antibody molecule exhibits a greater stability than an unmodified parent antibody molecule which selectively binds to the target.

In a second aspect of the invention, there is provided a nucleotide sequence encoding the modified antibody molecule according to the first aspect of the invention.

A third aspect of the invention provides an expression vector containing a nucleotide sequence encoding the modified antibody molecule according to the first aspect of the invention.

In a fourth aspect of the invention, there is provided a host cell producing a modified antibody molecule according to the first aspect of the invention, resulting from the expression of the nucleotide sequence encoding the modified antibody molecule.

A fifth aspect of the invention provides the modified antibody molecule according to the first aspect of the invention in association with at least one other agent.

A sixth aspect of the invention provides a composition comprising the modified antibody molecule according to the first aspect of the invention and a pharmaceutically acceptable carrier, excipient and/or diluent.

In a seventh aspect of the invention there is provided the use of a modified antibody molecule according to the first aspect of the invention in the manufacture of a medicament for the treatment and/or diagnosis and/or prevention of Cancer, inflammatory disorders such as Crohn's disease, rheumatoid arthritis, psoriasis, asthma and allergies; cardiovascular diseases such as restenosis, cardiopulmonary bypass, myocardial infarction; infectious diseases such as respiratory syncytial virus, HIV, hepatitis C; autoimmune disorders such as lupus and dermatomyosotis; Central Nervous System disorders such as Alzheimer's, transplant rejection and graft-versus-host disease; and nephritis, sepsis, haemoglobinuria, chemotherapy induced thrombocytopenia, and addiction (e.g. cocaine and nicotine).

In an eighth aspect of the invention, there is provided a phage containing the nucleotide sequence according to the second aspect of the invention.

### BRIEF DESCRIPTION OF THE DRAWINGS

FIG. 1 demonstrates the biophysical serum stability of scFv fragments. Immunoreactivity of wild-type scFv 4.10 (▪—solid squares), 22W (□ open squares) and mutant variants 4.9M (●—solid circles) and 22M (∘—open cicles) with MCF7 cells was measured by flow cytometry after incubating the constructs at 37° C. in human serum for various time points.

FIG. 2 shows the temperature dependent immunoreactivity of scFv mutant 4.9M. Binding of scFv 4.9M was analysed by flow cytometry before and after incubation at 37° C. for 1 h either in PBS (black bar) or human serum (HS; white bar).

FIG. 3 shows the thermal stability of mutated scFv 4.9M. Analytical size-exclusion FPLC on a calibrated Superdex 75 column was performed before (A) and after (B) incubation of 20 μg/ml scFv in PBS at 37° C. for 1 h. Monomeric protein eluted at 12.5 ml at a flow rate of 0.3 m/min.

FIG. 4 demonstrates the affinity of huHMFG1 scFv variants. Equilibrium-binding curves for wild-type scFv's 4.10W (▪—solid squares), 22W (□—open squares) and mutant variants 4.9M (●—solid circles) and 22M (∘—open circles) were determined by flow cytometry. Binding activity to MUC1⁺ MCF7 cells at indicated concentrations is expressed in % of maximal median fluorescence intensity (MFI). Measurements were performed in triplicate; standard deviations are shown as bars. Binding affinity constants (K_{d}) were determined by fitting the cell binding data to the non-linear regression model according to the Levenberg-Marquard method.

FIG. 5 demonstrates the size-exclusion chromatographic profile of diabody 4.9. Dimers of IMAC purified diabody 4.9 eluted at 11.2 ml on a calibrated Superdex 75 column at a flow rate of 0.3 ml/min in PBS/50 mM imidazole pH 7.4.

FIG. 6 shows the characterisation of purified diabody 4.9. (A) The equilibrium-binding curve and binding affinity constants (K_{d}) for diabody 4.9 was determined as described in legend to FIG. 4. Binding measurements to MCF7 cells at indicated concentrations was performed in triplicate and is expressed as median fluorescence intensity (MFI); standard deviations are shown as bars. (B) 20 μg/ml of diabody was incubated in human serum at 37° C. and immunoreactivity with MCF7 cells was determined at indicated time points.

FIG. 7 shows a whole cell ELISA of phage displayed wild-type and mutant scFv. Various concentrations of 4.10 wild-type scFv phage (▪—sold squares); 4.9 mutant scFv phage (●—solid circles) or M13KO7 helper phage (x) were incubated with subconfluent MCF7 cells grown in 96-well plates for 2 h at 37° C. Bound phage were detected with anti-M13-HRP as described in Material and Methods.

FIG. 8 demonstrates the sequence of stability engineered huHMFG1-scFv/Clone 4.9. Sequence shows restrictions sites, and labelled modification site. Silent modification V_{H}110 introduced to generate BstEII restriction site for convenient cloning. L10 silent modification introduced by PCR.

FIG. 9 is a schematic representation of fusion proteins used in this study. A) rapLR1-FB-huHMFG1-scFv 17W. B) rapLR1-FB-huHMFG1-scFv 17M, same construct as in A) but with the R71A mutation in the V_{H} domain. C) HMFG1-scFv 4.9M, V_{H}-V_{L} domain orientation, (GGGGS)₃ linker (SEQ ID NO: 10), V_{H} R71A mutation. D) rapLR1-(G₄S)-huHMFG1-scFv 4.9M, rapLR1 fused to the scFv shown in C via a GGGGS linker (SEQ ID NO: 7) (* denotes the R71A mutation in the V_{H} domain).

FIG. 10 is a schematic representation of the scfv-fusion protein expression vector pDD-1. Ap^{R}, ampicillin resistance gene; ColE1, origin of DNA replication; c-myc, sequence encoding the c-myc epitope; His₆, hexa-histidine encoding sequence; P/O, lac wild-type promoter/operator; rbs, ribosome binding site; pelB, signal peptide sequence of bacterial pectate lyase; V_{H}, variable heavy chain; V_{L}, rapLR1, Rana pipiens liver ribonuclease 1, variable light chain; H71, framework 3 residue 71; FB, fragment B of staphylococcal protein A (AKKLNDAQAPKSD) (SEQ ID NO: 11) and G₄S, spacer connecting the ribonuclease with the scFv, respectively; 17aa (ASSGGGGSGGGGSGGSA) (SEQ ID NO: 8) and (G₄S)₃, linker connecting the variable domains, respectively. Restriction sites for cloning are indicated.

FIG. 11 is a schematic representation of the scFv-fusion protein expression vector pBJ-2. Ap^{R}, ampicillin resistance gene; ColE1, origin of DNA replication; c-myc, sequence encoding the c-myc epitope; His₆, hexa-histidine encoding sequence; P/O, lac wild-type promoter/operator; rbs, ribosome binding site; pelB, signal peptide sequence of bacterial pectate lyase; V_{H}, variable heavy chain; VL, rapLR1, Rana pipiens liver ribonuclease 1, variable light chain; H71, framework 3 residue 71; FB, fragment B of staphylococcal protein A (AKKLNDAQAPKSD) and G₄S, spacer connecting the ribonuclease with the scFv, respectively; 17aa (ASSGGGGSGGGGSGGSA) (SEQ ID NO: 8) and (G₄S)₃, linker connecting the variable domains, respectively. Restriction sites for cloning are indicated.

FIG. 12 shows the SDS-polyacrylamide gel electrophoresis of fusion proteins. Lane 1, rapLR1-FB-huHMFG1-scFv 17W after chromatography on Ni²⁺-NTA-agarose; Lanes 2 and 3, rapLR1-FB-huHMFG1-scFv 17M and rapLR1-(G₄S)-huHMFG1-scFv 4.9M, respectively, after chromatography on SP-Sepharose.

FIG. 13 shows a cytotoxicity analysis. Cytotoxicity of rapLR1-FB-huHMFG1-scFv 17M, rapLR1-(G₄S)-huHMFG1-scFv 4.9M, and rapLR1 alone, to MCF7 cells. Cytotoxicity assays were performed by measuring the incorporation of [¹⁴C]leucine into synthesised cell proteins as described in Methods. rapLR1-FB-huHMFG1-scFv 17M, open circles; rapLR1-(G₄S)-huHMFG1-scFv 4.9M, solid circles; rapLR1, asterisks.

FIG. 14 is a schematic representation of the three huHMFG1 homologous models. Model-1 with the side chain of Arg71 pointing out toward the surface of the protein is in black, Model-2 with Arg71 buried inside between motifs H1 and H2 is in light grey, and Model-3 with Ala71 is in medium grey. The illustration was made using programs Bobscript (Esnouf (1997) Journal of Molecular Graphics & Modelling 15, 132-134, 112-113) and Raster3D (Merritt et al. (1997) Methods in Enzymology 277, 505-524).

FIG. 15 is a chart showing binding specificity of modified anti-MUC1 ScFv molecules.

### DETAILED DESCRIPTION OF THE INVENTION

In a first aspect of the invention there is provided a modified antibody molecule which selectively binds to a specific target, the antibody molecule being modified at at least one amino acid residue antigen binding selectivity and/or affinity, characterised in that the modified antibody molecule exhibits a greater stability than an unmodified parent antibody molecule which selectively binds to the target.

The stability of the antibody molecule can be measured and compared using the methods described in example 4 (incubation in human serum and flow cytometry analysis of binding).

Preferably the site of modification is at one or more of the corners of the inner faces of the beta-sheets of the antibody molecule. These beta sheets are constituent parts of the antigen binding site.

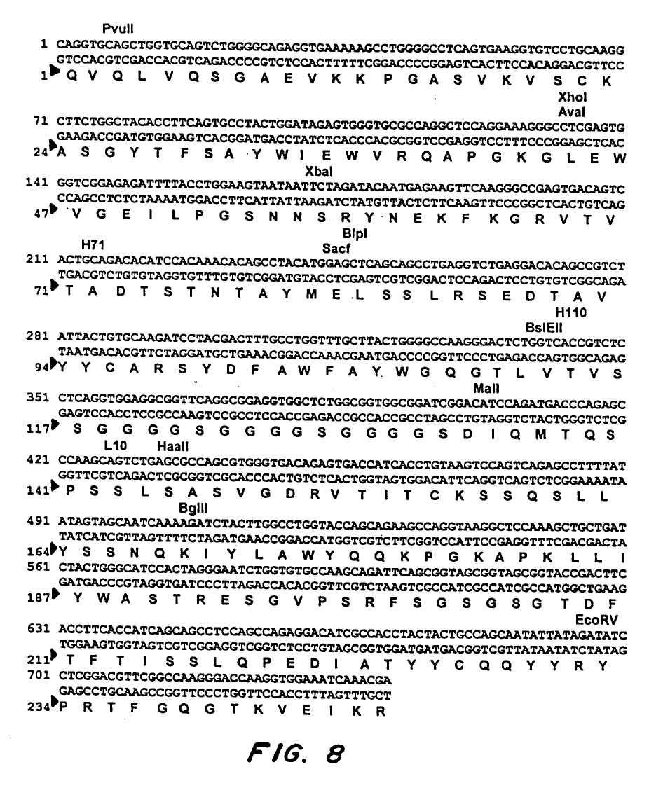

More preferably the modified antibody molecule is mutated at the amino acid residue V_{H}71 in the amino acid sequence of FIG. 8 (SEQ ID NO: 1) or a corresponding amino acid residue in other antibody molecules.

The position of amino acid residues corresponding to the V_{H}71 amino acid residue in the sequence of FIG. 8 (SEQ ID NO: 1) is identified by the KABAT numbering system. The same residue can be identified in any given antibody by submitting the Fv protein sequence online at http://www.bioinf.org.uk/abs/seqtest.html. The server for this site aligns the submitted sequence to all KABAT database entries and makes the accurate numbering of residues. Also, any “unusual” residues (i.e. occurrence at a given position <1%) are reported. The sequences can also be aligned manually according to the method of Kabat et al. (1991) Sequences of Proteins of Immunological Interest. NIH publication no. 91-3242. Conveniently the specific target is the MUC-1 gene product.

Preferably the modified amino acid residue possesses at least one physicochemical property that is different to the amino acid before modification.

The physicochemical properties of amino acids include charge; and/or hydrophobicity/hydrophilicity; and/or size; and/or structure; and/or volume and/or polarity and/or side chain characteristics (e.g. aromatic, aliphatic). For example, the size of the amino acid side chain can be reduced by modification from arginine to alanine.

The physicochemical properties of an amino acid can be measured using methods known in the art and from Bigelow (1967) J. Theor. Biol. 16, 187-211, Fauchere et al.(1988) Int. J. Peptide protein Res., 32, 269-278 and Goldsack and Chalifoux (1973) J. Theor. Biol. 39, 645-651

Preferably, the modification increases the buried surface of the antigen-binding site. Conveniently, the modification increases the buried surface area between motifs. The buried surface area can be measured using the method of creating computer homology models as described in example 7.

Conveniently the modified antibody molecule has a binding selectivity equivalent to gamma 1, kappa anti-HMFG monoclonal antibody (HMFG1).

Equivalence of binding selectivity can be measured using methods described in the examples, in particular Examples 2 (FACS analysis) and 5 (Phage ELISA).

Preferably the modified antibody molecule is a single chain antibody. Most preferably the single chain antibody is an ScFv or a diabody.

An improved affinity of binding was also found to be achieved if the mutant scFv was further engineered into a bivalent diabody. The diabody molecule can be expected to exhibit improved pharmacokinetics and tumour retention properties (Adams et al., Br J Cancer 77, 1405-1412, 1998). Notably, the diabody molecules generated by the inventors did not exhibit the sharp decline in immunoreactivity within one hour of serum incubation as observed for the scFv. Binding affinity can be measured using the methods of example 3 (affinity constant determination).

Conveniently the modified antibody molecule is humanised. Humanised antibodies are suitable for administration to humans without invoking an immune response by the human against the administered immunoglobulin. Humanised forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanisation can be performed following the method of Winter and co-workers (Jones et al., Nature, 321:522-525 (1986); Riechmann et al., Nature, 332:323-327 (1988); Verhoeyen et al., Science, 239:1534-15.36 (1988)). In some instances, Fv framework residues of the human immunoglobulin are replaced by corresponding non-human residues. Humanised antibodies can also comprise residues that are found neither in the recipient antibody nor in the imported CDR or framework sequences. In general, the humanised antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanised antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)). Preferably, a human amino acid residue is modified to the murine amino acid.

In a second aspect of the invention is provided a nucleotide sequence encoding the modified antibody molecule according to the first aspect of the invention. Preferably the nucleotide sequence is that of FIG. 8 (SEQ ID NO: 2).

A third aspect of the invention provides an expression vector containing a nucleotide sequence encoding the modified antibody molecule according to the first aspect of the invention. Preferably the nucleotide sequence is that of FIG. 8 (SEQ ID NO: 2).

The DNA is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. No. 4,440,859 issued 3 Apr. 1984 to Rutter et al., U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al., U.S. Pat. No. 4,678,751 issued 7 Jul. 1987 to Goeddel, U.S. Pat. No. 4,704,362 issued 3 Nov. 1987 to Itakura et al., U.S. Pat. No. 4,710,463 issued 1 Dec. 1987 to Murray, U.S. Pat. No. 4,757,006 issued 12 Jul. 1988 to Toole, Jr. et al., U.S. Pat. No. 4,766,075 issued 23 Aug. 1988 to Goeddel et al. and U.S. Pat. No. 4,810,648 issued 7 Mar. 1989 to Stalker, all of which are incorporated herein by reference.

The DNA encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Bacterial promoters include the E. coli lacI and lacZ promoters, the T3 and T7 promoters, the gpt promoter, the phage λ PR and PL promoters, the phoA promoter and the trp promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome binding site for translation. (Hastings et al., International Patent No. WO 98/16643, published 23 Apr. 1998)

The vector is then introduced into the host through standard techniques. Generally, not all of the hosts will be transformed by the vector and it will therefore be necessary to select for transformed host cells. One selection technique involves incorporating into the expression vector a DNA sequence marker, with any necessary control elements, that codes for a selectable trait in the transformed cell. These markers include dihydrofolate reductase, G418 or neomycin resistance for eukaryotic cell culture, and tetracycline, kanamycin or ampicillin resistance genes for culturing in E. coli and other bacteria. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

The polypeptide of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography.

Many expression systems are known, including systems employing: bacteria (e.g. E. coli and Bacillus subtilis) transformed with, for example, recombinant bacteriophage, plasmid or cosmid DNA expression vectors; yeasts (e.g. Saccharomyces cerevisiae) transformed with, for example, yeast expression vectors; insect cell systems transformed with, for example, viral expression vectors (e.g. baculovirus); plant cell systems transfected with, for example, viral or bacterial expression vectors; animal cell systems transfected with, for example, adenovirus expression vectors.

The vectors can include a prokaryotic replicon, such as the Col E1 ori, for propagation in a prokaryote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one pre-formed cohesive end.

Synthetic linkers containing a variety of restriction endonuclease sites are commercially available from a number of sources including International Biotechnologies Inc, New Haven, CN, USA.

Thus, in a fourth aspect of the invention there is provided a host cell producing a modified antibody molecule according to the first aspect of the invention, resulting from the expression of the nucleotide sequence encoding the modified antibody molecule. Preferably the nucleotide sequence is that of FIG. 8 (SEQ ID NO: 2).

In a fifth aspect, the invention provides the modified antibody molecule according to the first aspect of the invention in association with at least one other agent. Preferably the agent is at least one selected from drugs, toxins (e.g. PE, DT, Ricin A etc.), radionuclides (e.g. ⁹⁰Y, ¹³¹I, ¹²⁵I, ^{99m}Tc etc.), nucleases (e.g. RNases, caspases and DNases), enzymes, cytokines and chemokines.

Examples of the use of antibodies in association with other agents is described in GB 2 360 772 and GB 2 383 538. More preferably the agent is conjugated to the modified antibody molecule. Most preferably the agent is a nuclease.

A sixth aspect of the invention provides a composition comprising the modified antibody molecule according to the first aspect of the invention and a pharmaceutically acceptable carrier, excipient and/or diluent. Preferably the composition comprises the nucleotide sequence as described in the second aspect of the invention.

More preferably the composition further comprises at least one other agent. Most preferably the agent is at least one selected from drugs, toxins (e.g. PE, DT, Ricin A etc.), radionuclides (e.g. ⁹⁰Y ¹³¹I, ¹²⁵I, ^{99m}Tc etc.), nucleases (e.g. RNases, caspases and DNases), enzymes for prodrug approaches, cytokines, chemokines.

In a seventh aspect of the invention there is provided the use of a modified antibody molecule according to the first aspect of the invention in the manufacture of a medicament for the treatment and/or diagnosis and/or prevention of Cancer, inflammatory disorders such as Crohn's disease, rheumatoid arthritis, psoriasis, asthma and allergies; cardiovascular diseases such as restenosis, cardiopulmonary bypass, myocardial infarction; infectious diseases such as respiratory syncytial virus, HIV, hepatitis C; autoimmune disorders such as lupus and dermatomyosotis; Central Nervous System disorders such as Alzheimer's, transplant rejection and graft-versus-host disease; and nephritis, sepsis, haemoglobinuria, chemotherapy induced thrombocytopenia, and addiction (e.g. cocaine and nicotine).

Preferably the disease is Cancer. More preferably the cancer is a cancer of the Breast, Ovary, Uterus, Lung, B-cell non-Hodgkins lymphoma (B-NHL), multiple myeloma, acute myelogenous leukemia (AML), chronic lymphocytic leukemia (CLL) and hairy cell leukemia.

In an eighth aspect of the invention there is provided a phage containing the nucleotide sequence according to the second aspect of the invention.

Preferably the nucleotide sequence is operably linked to the nucleotide sequence encoding a phage surface protein. More preferably the nucleotide sequence is expressed by the phage and displayed on the phage surface.

The invention further provides the use of the phage described above in a screening assay. Such a screening assay would identify mutant antibodies, antibody fragments or antibody derivatives displayed on phage that were able to bind a selective target. Preferably that target is the MUC-1 gene product.

The display of proteins and polypeptides on the surface of bacteriophage (phage), fused to one of the phage coat proteins, provides a powerful tool for the selection of selective ligands. This ‘phage display’ technique was originally used by Smith in 1985 (Science 228, 1315-7) to create large libraries of peptides for the purpose of selecting those with high affinity for a particular antigen. More recently, the method has been employed to present antibodies at the surface of phage in order to identify ligands having desired properties (McCafferty et al., Nature, 1990, 552-554).

The use of phage display to isolate ligands that bind biologically relevant molecules has been reviewed in Felici et al. (1995) Biotechnol. Annual Rev. 1, 149-183, Katz (1997) Annual Rev. Biophys. Biomol. Struct. 26, 27-45 and Hoogenboom et al. (1998) Immunotechnology 4(1), 1-20. Several randomised combinatorial peptide libraries have been constructed to select for polypeptides that bind different targets, e.g. cell surface receptors or DNA (reviewed by Kay, 1995, Perspect. Drug Discovery Des. 2, 251-268; Kay and Paul, 1996, Mol. Divers. 1, 139-140). Proteins and multimeric proteins have been successfully phage-displayed as functional molecules (see EP 0349578A, EP 0527839A, EP 0589877A; Chiswell and McCafferty, 1992, Trends Biotechnol. 10, 80-84). In addition, functional antibody fragments (e.g. Fab, single chain Fv [scFv]) have been expressed (McCafferty et al., 1990, Nature 348, 552-554; Barbas et al., 1991, Proc. Natl: Acad. Sci. USA 88, 7978-7982; Clackson et al., 1991, Nature 352, 624-628), and some of the shortcomings of human monoclonal antibody technology have been superseded since human high affinity antibody fragments have been isolated (Marks et al., 1991, J. Mol. Biol. 222, 581-597; Hoogenboom and Winter, 1992, J. Mol. Biol. 227, 381-388). Further information on the principles and practice of phage display is provided in Phage display of peptides and proteins: a laboratory manual Ed Kay, Winter and McCafferty (1996) Academic Press, Inc ISBN 0-12-402380-0, the disclosure of which is incorporated herein by reference.

The display of mutant antibody fragments on phage provides methods to detect selective binding of a polypeptide e.g. MUC-1 gene product to the test ScFV being expressed.

Meanings of Terms Used

The term “antibody molecule” shall be taken to refer to any one of an antibody, an antibody fragment, or antibody derivative. It is intended to embrace wildtype antibodies, synthetic antibodies, recombinant antibodies or antibody hybrids, such as, but not limited to, a single-chain modified antibody molecule produced by phage-display of immunoglobulin light and/or heavy chain variable and/or constant regions, or other immunointeractive molecule capable of binding to an antigen in an immunoassay format that is known to those skilled in the art.

The term “antibody derivative” refers to any modified antibody molecule that is capable of binding to an antigen in an immunoassay format that is known to those skilled in the art, such as a fragment of an antibody (e.g. Fab or Fv fragment), or a modified antibody molecule that is modified by the addition of one or more amino acids or other molecules to facilitate coupling the antibodies to another peptide or polypeptide, to a large carrier protein or to a solid support (e.g. the amino acids tyrosine, lysine, glutamic acid, aspartic acid, cysteine and derivatives thereof, NH₂-acetyl groups or COOH-terminal amido groups, amongst others).

The term “ScFv molecule” refers to any molecules wherein the V_{H} and V_{L} partner domains are linked via a flexible oligopeptide.

The term “diabody” refers to any molecules that are non-covalently associated dimers, in which each chain comprises two domains consisting of V_{H} and V_{L} domains. Both domains are connected by a linker that is too short to allow pairing between domains of the same chain. Thus, each chain alone is not capable of binding antigen, but two chains will assemble to dimeric diabodies with two functional antigen binding sites.

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). It is contemplated that where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “probe” and “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridisation procedures or microarray procedures to identify or amplify identical or related parts of mRNA or DNA molecules. A fragment or segment may uniquely identify each polynucleotide sequence of the present invention.

The terms “polypeptide” or “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. To be active, any polypeptide must have sufficient length to display biological and/or immunological activity.

The term “variant” (or “analogue”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimising the number of amino acid sequence changes made in regions of high homology (conserved regions) or by replacing amino acids with consensus sequence.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. Coli, will be free of glycosylation modifications; polypeptides or proteins expressed in yeast will have a glycosylation pattern in general different from those expressed in mammalian cells.

The term “expression vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The terms “selective binding” and “binding selectivity” indicates that the variable regions of the antibodies of the invention recognise and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding selectivity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognise and bind fragments of the polypeptides of the invention are also contemplated, provided that the antibodies are first and foremost selective for, as defined above, full-length polypeptides of the invention. As with antibodies that are selective for full length polypeptides of the invention, antibodies of the invention that recognise fragments are those which can distinguish polypeptides from the same family of polypeptides despite inherent sequence identity, homology, or similarity found in the family of proteins.

The term “binding affinity” includes the meaning of the strength of binding between an antibody molecule and an antigen.

The present invention will be further understood by reference to the following non-limiting examples.

### EXAMPLES

### Example 1

### Generating Antibody Derivatives

Identification of unusual framework residues The identification of non matching “key residues” (Chothia, C. et al. (1989) Nature 342:877) within the human variable domain acceptor antibody framework regions of huHMFG1 and the murine variable domain donor antibody framework regions of HMFG1 was undertaken by manual inspection.

The Chothia canonical-class assignments of the complementarity determining regions (L1-L3, H1 and H2) of the donor antibody, were determined by screening the sequence against sequence templates of antibody repertoires (http://www.bioinf.org.uk/). Amino acid residues at the V_{H}/V_{L} interface (Chothia et al., 1985, JMB 186, 651-663) were also inspected so as to identify unusual residues with the potential to interfere with V_{H}-V_{L} inter-domain stability.

Generation of Wild Type scFv huHMFG1, scFv Mutants and Diabody

The huHMFG1 variable light chain was PCR amplified from plasmid pAS 1 (Antisoma Ltd). Plasmid pAS1 contains the full light chain of the humanised antibody. The primers used for PCR amplification were huHMFG1V_{L} (forward):

| | |
|---|---|
| 5′ CATGCCATGGCCGACATCCAGATGACC 3′ | (SEQ ID NO:3) |

and huHMFG1 V_{L} (back):

| | |
|---|---|
| 5′ CCGCTCGAGGCTCGTTTGATTTCCAC 3′. | (SEQ ID NO:4) |

These primers were designed to incorporate flanking NcoI and Xho I restriction sites (underlined) for cloning into the vector pHEN2 (MRC, Cambridge, UK). The NcoI site was incorporated into the forward primer and the Xho I site was incorporated into the reverse primer.

The variable heavy chain was PCR amplified from plasmid pAS2 (Antisoma Ltd). Plasmid pAS2 contains the full heavy chain of the humanised antibody. Primers huHMFG1V_{H} (forward):

| | |
|---|---|
| 5′ GTAGTGCAGAGGTGCAGCTGGTG 3′ | (SEQ ID NO: 5) |

and huHMFG1V_{H} (back):

(SEQ ID NO:6)

5′ TTAGCGGCCGCGGATCCTGAGGAGACTGTGAC 3′

NO: 6)
were used for PCR amplification. These primers were designed to incorporate flanking ApaLI and Not I restriction sites (underlined) for cloning into the vector pHEN2 (MRC, Cambridge, UK). The ApaLI site was incorporated into the forward primer and the Not I site was incorporated into the reverse primer.

ScFv variants in either domain orientation and different linker peptide lengths possessing the flanking NcoI and BamHI restriction sites were generated by PCR using the scFv wild type DNA as a template. Modifications were introduced by oligonucleotide directed mutagenesis and overlap extension PCR techniques as described in the art e.g. Ho, S. N., et al. (1989) Gene 77:51.

The diabody was constructed in the V_{H}-V_{L} orientation by shortening the linker to 5 residues (GGGGS) (SEQ ID NO: 7). This shortening was produced using the mutant scFv as a template for PCR as described in the art e.g. Holliger, P. et al.(1993) Proc. Natl. Acad. Sci. USA 90:6444.

Fragments were cloned into vector pHOG21 (Kipriyanov, S. M. et al. (1996) J Immunol Methods 196:51) for expression of soluble protein.

Periplasmic Expression and Purification of scFv Fragments

The E. coli strain TG1 (Stratagene, La Jolla, Calif.), was transformed with the scFv expression plasmid, and grown at 37° C. and 230 rpm in 1000 ml 2YT culture medium (Difco) containing 100 μg/ml ampicillin and 100 mM glucose (2YT_{GA}).

The E. coli cells were then pelleted by centrifugation after having reached an optical density (OD₆₀₀) of 0.8-1.0 at 1500 g for 20 min at 20° C. the pelleted cells were re-suspended in 1000 ml of fresh 2YT culture medium containing 100 μg/ml ampicillin, 0.4 M sucrose and 1 mM IPTG. The scFv fragments were expressed by the cells at 18° C. and 18-20 h induction.

The E. coli cells were then pelleted by centrifugation at 7000 g, 30 min at 4° C. The cells were re-suspended in 5% of the initial volume in a periplasmic extraction buffer (50 mM Tris, 1 mM EDTA, 20% Sucrose, pH 8.0) and incubated for 1 h on ice.

The cell suspension was then centrifuged at 30,000 g at 4° C. for 1 h. The supernatant containing the soluble scFv was thoroughly dialysed against SP10 buffer (300 mM NaCl, 50 mM NaH₂PO₄, 10 mM imidazole, pH 8.0).

The dialysed crude periplasmic extract was purified by immobilised metal affinity chromatography (IMAC) using Ni-NTA columns according to the protocol of the manufacturer (Qiagen, Valencia, Calif.). The purified scFv antibody fragments were then eluted and extensively dialysed against PBS, 50 mM imidazole.

The monomeric scFv antibody fragments were separated from higher molecular forms by size-exclusion chromatography using a calibrated Superdex 75 HR 10/30 column (Amersham Pharmacia, Piscataway, N.J.).

The monomeric scFv fractions were then analysed on 4-20% SDS-PAGE under reducing conditions and stained with a protein stain e.g. Simply Blue™ Safe Stain (Invitrogen, Carlsbad, Calif.). Alternatively the protein bands can be visualised by the Western blot method utilising an anti-c-myc mAb 9E10 (Roche, Indianapolis, Ind.) as the primary antibody, and an anti-mouse IgG conjugated to alkaline phosphatase (Sigma, St. Louis, Mo.) as secondary antibody.

Concentrations of monomeric scFv fractions were determined spectrophotometrically from the absorbance at A_{280 nm} using the extinction coefficient ε^{1 mg/ml}=1.67.

Results

Generation and Characterisation of Wild Type Sequence scFvs

A small antigen binding molecule was generated as a building block for subsequent construction of recombinant fusion proteins by re-cloning the variable light and heavy chains of the humanised mAb huHMFG1 into a V_{L}-V_{H} oriented scFv format. The heavy and light chains were separated by a synthetic 17 amino acid linker peptide (clone 17W, Table 1).

Clone 17W showed selective binding to MUC1⁺ tumour cells with a half-life in human serum at 37° C. of less than two hours (data not shown).

In order to determine whether the linker peptide connecting the V_{L}-V_{H} domains accounted for the poor stability of the wild type scFv two further variants, 22W and 4.10W, were generated (Table 1) possessing synthetic linker peptides of lengths of 22 and 15 amino acids, respectively. The 22W and 4.10W clones exhibited selective binding to MUC1⁺ tumour cells. However, neither the 22W or 4.10W clone possessed an increase in their half lives (data not shown).

TABLE 1
huHMFG1 scFv variants

| | | | | |
|---|---|---|---|---|
| Clone | | Orienta- | Linker | |
| V_{H} 71R | V_{H} 71A | tion | length | Linker sequence |

| | | | | |
|---|---|---|---|---|
| 17 W | 17 M | V_{L} − V_{H} | 17 | ASSGGGGSGGGGSGGSA |
| | | | | (SEQ ID NO: 8) |
| 22 W | 22 M | V_{L} − V_{H} | 22 | ASSGGGGSGGGGSGGSAGGGGS |
| | | | | (SEQ ID NO: 9) |
| 4.10 W | 4.9 M | V_{H} − V_{L} | 15 | GGGGSGGGGSGGGGS |
| | | | | (SEQ ID NO: 10) |

Analysis of huHMFG1 Variable Domain Wild-Type Sequence

The amino acid sequence of the humanised scFv antibody was analysed as described in Example 1. Amino acid position V_{H}71 was identified to be potentially critical for maintaining the structural integrity of the wild-type scFv fragment antigen binding site when grafted into a non-wild-type framework.

The effects of residue V_{H}71 on the stability and antigen binding of the humanised scFvs were studied in three mutant variants generated by the replacement of the arginine from the human framework 3 sequence with an alanine from the murine donor antibody (clones 17M, 22M, 4.9M; Table 1).

### Example 2

### Antibody Fragment Binding

The selective binding of the constructs was determined by flow cytometry using the human MUC1⁺ cell lines MCF7 (ATCC # HTB-22) and SKOV-3 (ATCC # HTB-77) Mouse myeloma B cell line Sp2/0-Ag14 (ATCC # HTB-77) was used as a negative control. 5×10⁵ cells from each of the test cell lines were incubated with 100 μl of a sample containing either the scFv fragments, or control antibodies, in FACS buffer (PBS, 0.1% NaN3, 2% FBS) for 45 min at 4° C. in round bottom 96-well microtitre plates.

The test cells were pelleted at 200 g at 4° C. for 5 min and washed twice with 200 μl FACS buffer. For detection of antibodies bound to the test cells, the cells were first incubated for 30 min at 4° C. with saturating concentrations of the anti-c-myc mAb 9E10 (10 μg/ml; Roche), followed by two washes and incubation with saturating amounts of FITC-labelled anti-mouse IgG (13 μg/ml; Jackson Immuno Research, West Grove, Pa.) for 30 min at 4° C.

In order to exclude dead cells from the analysis, the cells were washed twice with 200 μl FACS buffer and then re-suspended in FACS buffer containing 10 μg/ml propidium iodide (Sigma). Background fluorescence was determined by using cells incubated with 9E10 antibody and FITC-labelled anti-mouse antibody under the same conditions.

Stained cells were analysed on a FACScan Flow Cytometer (BD Bioscience, San Jose, Calif.), and median fluorescence intensity (MFI) was calculated using the CellQuest™ software (BD Bioscience).

### Example 3

### Antibody Derivative Affinity Constants (K_{d}) Determination

Affinity measurements were performed as previously described in the art e.g. Benedict, C. A. et al. (1997) J Immunol Methods 201:223 with the following modifications: Varying concentrations of antibodies (or antibody fragments) were incubated in triplicate with 5×10⁵ MCF7 cells at room temperature in FACS buffer for two hours. Bound antibodies were detected under the same conditions, as described in the section entitled binding assays above.

After two final washing steps in 200 μl of FACS buffer, the cells were fixed in PBS buffer containing 2% paraformaldehyde for 15 minutes at room temperature and analysed by flow cytometry.

The median fluorescence intensity (MFI) was determined as described in the binding assays section above and the background fluorescence was subtracted. Equilibrium constants were determined by using the Marquardt-Levenberg algorithm for non-linear regression with the GraphPad Prism version 3.0a for Macintosh (GraphPad Software, San Diego, Calif.).

Results

Affinity constants of wild-type sequence scFvs in either V_{L}-V_{H} (clone 22W, Table 1) or opposite orientation (clone 4.10W, Table 1) and mutant variants (22M, 4.9M; Table 1) were determined. As shown in FIG. 4 mutagenesis of V_{H}71Arg to Ala only had a moderate effect on binding affinity of the constructs to the target cells irrespective of the variable domain orientation. Since a temperature dependent decrease in immunoreactivity of the mutant scFv fragments with rapid onset was observed in the stability assay the affinity constant of the construct with the tightest antigen binding (clone 4.9M) was again determined after pre-incubation of the construct in PBS for 1 hour at 37° C. This short exposure to physiological body temperature resulted in 1.9-fold decreased antigen binding affinity (Table II).

### Example 4

### Stability & Specificity

The ScFv fragments or diabodies were incubated at a concentration of 20 μg/ml in 90% human serum at 37° C. for up to seven days. Samples were taken at multiple time points and stored at −20° C. The binding activity of each of the samples to MUC1⁺ MCF7 cells was determined by flow cytometry. The MFI was determined as described in the above.

The temperature-dependent degradation of monomeric scFvs was determined by incubation of samples at 37° C. in 90% PBS at a concentration of 20 μg/ml for 1 h, followed by analytical gel filtration.

Effects of Modification V_{H}71(Arg to Ala) on Stability and Specificity of scFv Fragments

For initial characterisation the mutant variant scFvs (clones 17M, 22M, 4.9M; Table 1) were IMAC purified. During flow cytometry analysis all variants showed selective binding to MUC1⁺ MCF7 cells.

Stability of the engineered scFvs was assessed by incubating the ScFv molecule in human serum at 37° C. for 12 hours. The half live of mutant variant 17M was not increased when compared with the wild-type scFv 17W.

Monomers of the mutant scFvs 22M and 4.9M, as well as their wild-type counterparts (22W and 4.9W) were separated from higher molecular weight species and bacterial contaminants by size exclusion chromatography to yield a protein with >95% purity.

Both purified wild-type and mutant scFvs exhibited specific binding towards MUC1⁺ tumour cells as shown in FIG. 15, for the constructs in the VH-VL orientation.

The stability of purified monomers was again determined by incubation of the fragments in human serum at 37° C. for multiple different time points. FIG. 1 shows that both the 22M and 4.9M mutant scFvs exhibited dramatically improved stability when compared with the wild type sequence constructs. In fact, both wild type variants (22W and 4.10W) exhibited a drop of MFI to almost 0% within 24 hours, whereas both of the two mutant variants (22M and 4.9M) continued to exhibit an MFI of over 25% at 168 hours.

Both mutant variants did exhibit a marked decline in cell binding activity within the first hour of serum incubation (4.9M 39.8%; 22M 50.3%). The increase in stability does, however, allow a retention of binding activity to tumour cells even after seven days of serum incubation (FIG. 1, Table II).

The effect of whether elevated temperature or serum components play a role in inducing the reduction in binding activity was further investigated by incubating a sample of mutant scFv 4.9M in PBS for 1 h at 37° C. and measuring the binding activity to MCF7 cells by flow cytometry.

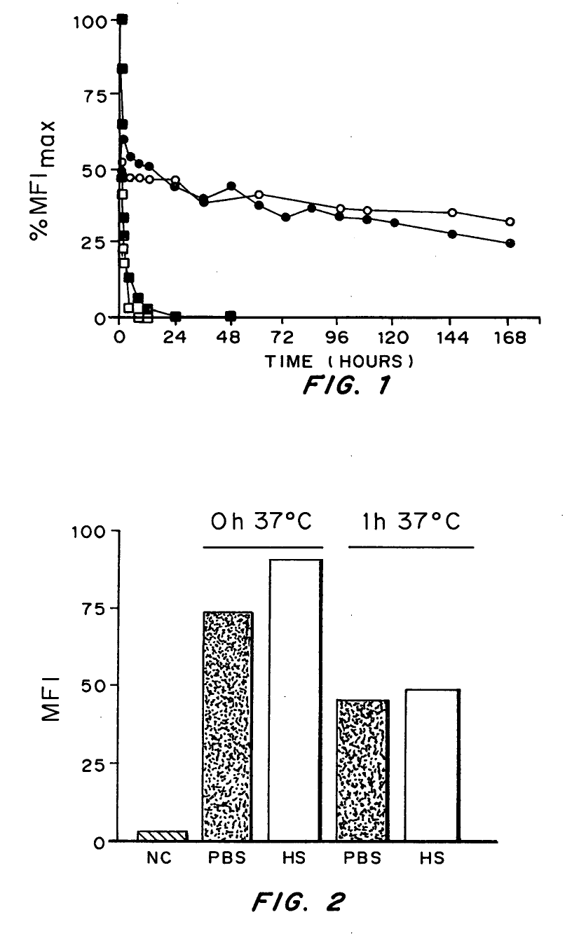

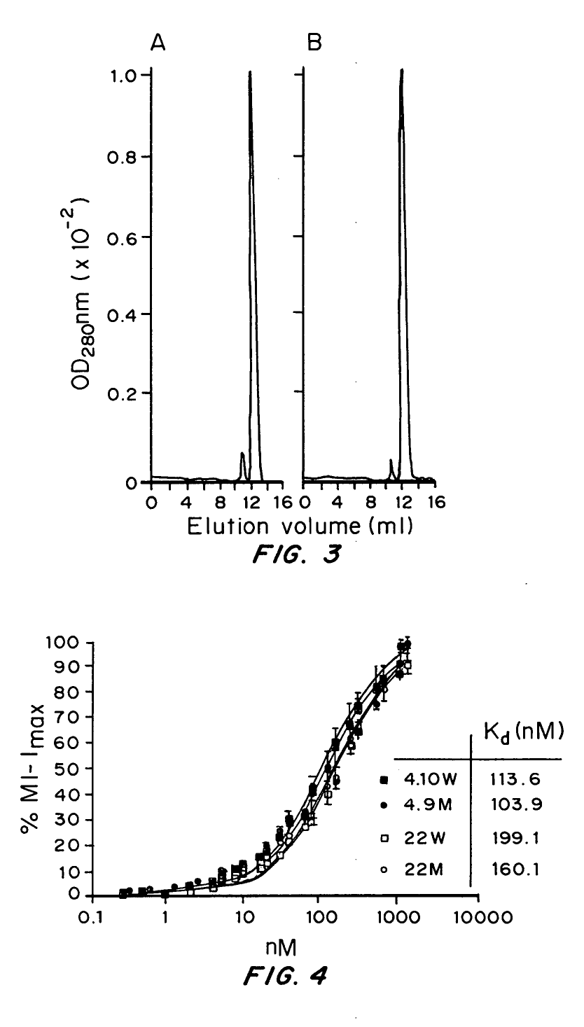

FIG. 2 shows the median fluorescence intensity decreased to a similar extent when the scFv was incubated in both PBS and human serum at 37° C. However, this was not due to temperature dependent degradation of the monomeric protein at this time point as shown by analytical size exclusion chromatography (FIG. 3).

Hence, this confirms that the initial decrease in immunoreactivity is due to a reduced binding affinity in the mutant clones.

TABLE 2
Influence of temperature on stability and
affinity of mutant scFv 4.9M and diabody
Db 4.9M

| | | | Binding | |
|---|---|---|---|---|
| | | | affinity | |
| Incubation | Immunoreactivity | | constant | |
| at 37° C. | (% MFImax) | | (nM) | |
| (h) | scFv | Diabody | scFv | diabody |
| 0 | 100 | 100 | 103 | 8 |
| 1 | 60 | 100 | 195 | n.d |
| 8 | 52 | 100 | | |
| 24 | 44 | 50 | | |
| 72 | 34 | 6.5 | | |
| 168 | 25 | 4 | | |

n.d., not determined.

### Example 5

### Expression of scFv Fragments on Phage

Full length wild-type and mutant scFv encoding gene segments were cloned into phagemid pCANTAB-5 (Amersham Pharmacia, Uppsala, Sweden) after introduction of the SfiI and NotI flanking restriction sites by PCR. Phagemid virions were prepared essentially according to standard methods as described in the art (e.g. Sambrook & Russell (2001) Molecular Cloning 18.115 to 18.122).

In brief, phagemid-containing bacteria were grown in 50 ml 2YT_{GA} medium and infected with helper phage M13KO7 at a multiplicity of infection (M.O.I.) of 1:20 at mid log phase (at an optical density of OD₆₀₀ of 0.6-0.8) followed by incubation for 30 min at 37° C. The phagemid-containing bacteria were centrifuged (3000 g, room temperature, 15 min) and re-suspended in 300 ml of 2YT medium containing 100 μg/ml ampicillin, 25 μg/ml kanamycin, and no glucose.

Phagemid infected bacteria were grown overnight at 30° C. at 230 rpm in a bacterial shaker. The supernatant was clarified by centrifugation (3000 g, 4C.°, 15 min) and phagemid virions were purified by two consecutive precipitations on ice for 30 min with a solution containing 20% PEG/2.5 M NaCl.

Phage were then pelleted by centrifugation at 15,000 g, 4° C., for 30 min. The supernatant was discarded and the phage were re-suspended in PBS. After filtration through 0.45 μm syringe filters (Millipore), the phage were stored at 4° C. The titre of amplified phage was determined by infecting TG1 bacteria (Stratagene, La Jolla, Calif.) at logarithmic growth phase with different dilutions of phage and incubation for 30 min at 37° C. Aliquots of mixtures were spread on agar plates containing 2YT_{GA}. After overnight growth the number of colonies were counted.

Phage ELISA

The ability of phage displayed scFvs for recognising the target antigen MUC-1 was determined by whole cell ELISA with MCF-7 (ATCC # HTB-22).

The ELISA method uses enzymes which give a coloured reaction product, usually in solid phase assays. Enzymes such as horse radish peroxidase and phosphatase have been widely employed. A way of amplifying the phosphatase reaction is to use NADP as a substrate to generate NAD which now acts as a coenzyme for a second enzyme system. Pyrophosphatase from E. coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great selectivity and affinity.

1×10⁴ cells/well were seeded in a 96 well plate (Nunc Nalgene, Denmark) and grown in DMEM medium (Invitrogen, Carlsbad, Calif.) supplemented with 10% FBS until 60-70% confluency was reached.

Cells were fixed by the addition of paraformaldehyde diluted in PBS buffer at a final concentration of 1% and incubated for 10 min at room temperature. After washing with PBS buffer, a serial dilution of purified scFv phagemid virion or the M13KO7 helper phage (Invitrogen) alone, as a control, was added to the cells. The plates were gently shaken for 2 h at 37° C.

After 6 rounds of washing with a solution of PBS buffer/0.02% Tween-20 buffer, the rabbit anti-M13-HRP polyclonal antibody (1:5000, Amersham Pharmacia Biotech Inc., Piscataway, N.J.) was added to the plates. This mixture was then incubated at 37° C. for 1 hour. Cells were washed with PBS and bound phage scFvs were detected using ABTS (Sigma Chemicals, Saint Louis, MS) as a substrate. After 30 minutes of incubation, the absorbance was read with an ELISA reader (Bio-Tek Instruments Inc.) at 405 nm.

Results

Expression of scFv Variants on Phase

The use of the humanised scFv antibody as a template for selecting entire human fragments from phage display libraries by chain shuffling was investigated in both wild-type sequence scFv 4.10W and mutant 4.9M. These two clones were re-cloned into a phagemid vector for display on phage. The titre of the concentrated phage stock was 1×10^{12-1×10}¹³ cfu/ml.

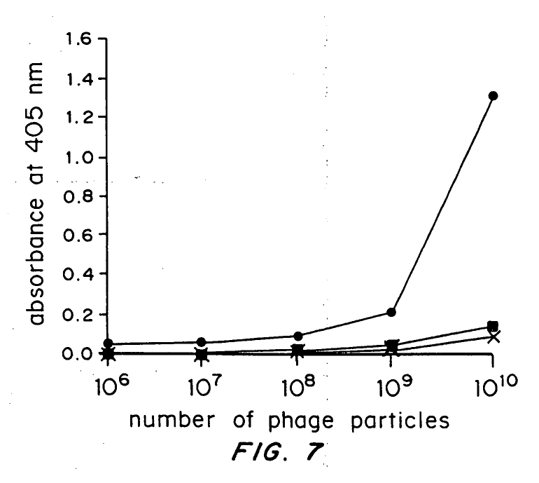

In whole cell ELISA, only the displayed mutant scFv 4.9M showed reactivity with MUC1⁺ tumour cells in a concentration dependent manner (FIG. 7). This indicates that the wild-type scFv was not appreciably expressed by and displayed on phage.

### Example 6

### Diabody

A dimeric diabody was generated from variant 4.9M by shortening the linker to 5 amino acid residues in order to prevent the pairing of the variable domains on the same polypeptide chain while still allowing the non-covalent association of two molecules to form a bivalent dimer (Holliger, P., et al. (1993) Proc. Natl. Acad. Sci. USA 90:6444).

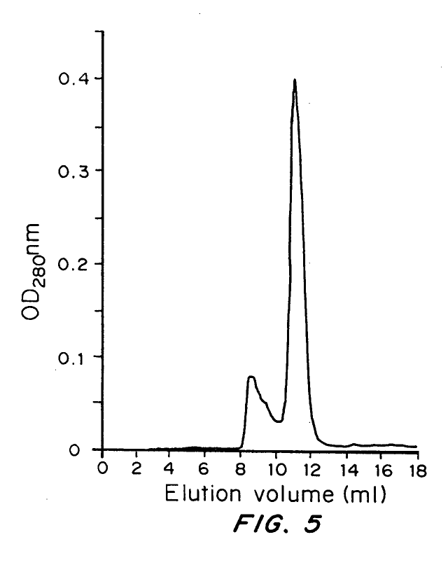

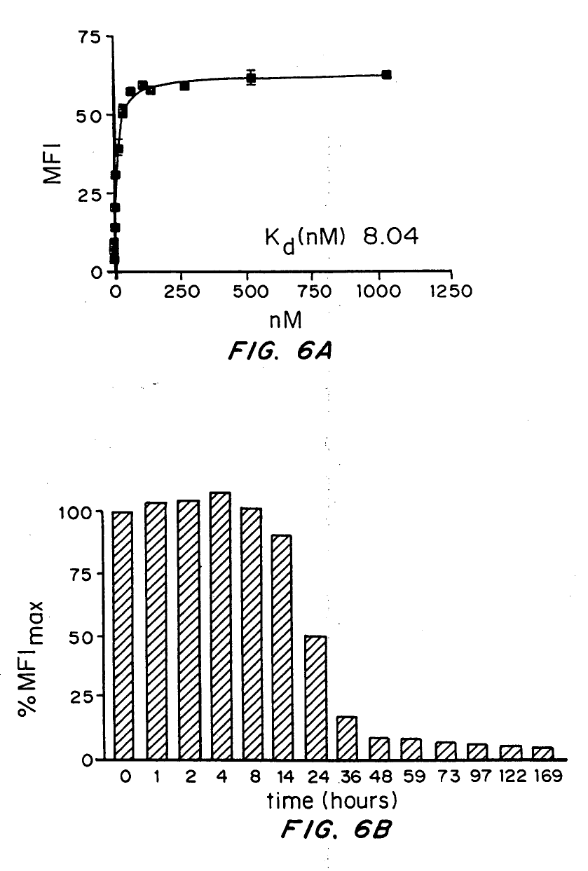

The soluble protein was purified by IMAC and dimers were separated to >95% purity from a small fraction of aggregates by size exclusion chromatography for further characterisation (see FIG. 5). The affinity of the diabody construct, designated Db 4.9M, was 13-fold higher than that of the corresponding scFv 4.9M (FIG. 6A, Table II).

The diabody remained highly stable for more than 12 hours in human serum at 37° C. and only reached its half life after 24 hours with rapid onset (FIG. 6B, Table II). Furthermore, the diabody did not exhibit any markedly impaired immunoreactivity with tumour cells after 1 hour of serum incubation at 37° C. (FIG. 6B).

### Example 7

### Computer Homology Model

To study the stabilising effect of the introduced modification V_{H}71-Arg to Ala, a computer homology model was generated.

Generation of Homologous Computer Models

The scFv huHMFG1 consists of the VH and VL domains with a 15 amino acid linker [GGGGSGGGGSGGGGS] (SEQ ID NO: 10) connecting the C-terminus of VH to the N-terminus of VL. Three homologous models of huHMFG1 were generated based on existing structures of scFv proteins with either an Arg or an Ala at position H71, which is in the Fr3 region of VH.

Two forms of huHMFG1-Arg71 were modelled with distinct conformations in their H2 and Fr3 regions based on observed conformations in the scFv structures with an Arg at position H71. In one form, the side chain of Arg71 is pointing out towards the surface of the protein. For example, see PDB entry 1NQB (Pei, et al. (1997) Proc Natl Acad Sci USA 94, 9637-9642), whereas in the other, this side chain is buried inside between motifs H1 (residues 31-35) and H2 (residues 50-65) For example, see PDB entry 2FBJ (Suh et al. (1986). Proteins 1, 74-80).

Only one form of huHMFG1-Ala71 was modelled because all the scFv structures with an Ala at H71 exhibit the same conformation For example, see PDB entry 1EO8 (Fleury et al. (2000). Proteins 40, 572-578), which is also similar to the scFv-Arg71 form with the side chain of Arg71 pointing out.

Model-1 with the side chain of Arg71 pointing out was built based on a trimeric antibody PDB entry 1NQB (Pei, et al. (1997) Proc Natl Acad Sci USA 94, 9637-9642), of which the sequence is 63% identical with that of huHMFG1. The starting model of huHMFGI was constructed with residues C121-C233 of 1NQB and C1-C120 of its symmetry mate. Mutations were performed according to the sequence of huHMFG1 using the program 0 (Jones et al. Methods in Enzymology 277, 173-208). followed by an energy minimisation of 200 cycles using program CNS (Brunger et al. (1997) Methods in Enzymology 277, 243-269).

Model-2 with the side chain of Arg71 buried inside was built based on the crystal structure of J539 antibody PDB entry 2FBJ (Suh et al. (1986). Proteins 1, 74-80). The sequence similarity of J539 with huHMFG1 is very low. Hence fragments including Fr3, H3, and V_{L} regions were taken from Model-1.

The remaining mutations were performed followed by energy minimisation as stated above. Model-3 with Ala71 was built based on PDB entry 1EO8.

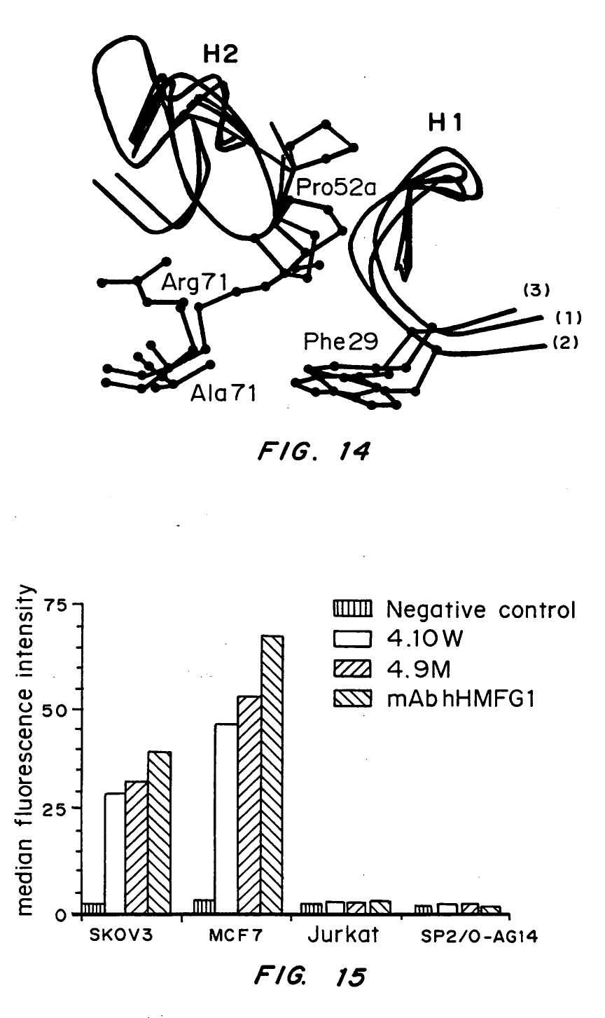

The V_{H} and V_{L} sequences of 1EO8 are 72 and 26% identical with those of huHMFG1, respectively. Mutations were performed in the V_{H} region; the V_{L} region was taken from Model-1. The model was optimised with 200 cycles of energy minimisation as stated above. FIG. 14 depicts the alignment of residue H71 and motifs H1 and H2 in the three huHMFG1 models.

Computer Homologous Modelling

HuHMFG1 is an scFv with an H2 region containing four residues. The conformations of Fr3 (H71 region) and H2 loop are similar in Model-1 and -3, whereas Model-2 has a different conformation as compared to Model-1 and -3 (FIG. 14).

In order to understand the structural consequences caused by the mutation of Arg71 to Ala71 in huHMFG1, we analysed the impact of this mutation on the conformation and relative position of motifs H1 and H2 by calculating the buried surface area of the interface between the two motifs for the three huHMFG1 models.

The results show a larger buried surface area for the Ala71 model (481A²) compared to that for the two Arg71 models (384 A² in Model-1 and 398 A² in Model-2).

It is believed that a larger buried surface area in huHMFG1-Ala71 (˜20% more than huHMFG1-Arg71) results in the stabilisation of the scFv. In order to validate this hypothesis, three homologous models of scFv huHMFG1 were built, including Model-1 with the side chain of Arg71 pointing out, Model-2 with the side chain of Arg71 buried inside, and Model-3 with Ala71. The models reveal a significantly larger buried surface area between motifs H1 and H2 for the Ala71 form, which is ˜20% more than that for both Arg71 forms. This larger buried surface area between H1 and H2 is responsible for the profound stability of huHMFG1-Ala71.

### Example 8

### Association with Other Agents

The antibodies, antibody fragments and antibody derivatives of the invention may be conjugated to the other agents that they are associated with. Such immunoconjugates include an antibody conjugated to a cytotoxic agent (e.g. a chemotherapeutic agent) or a radioactive isotope.

Enzymatically active toxins and fragments thereof that can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ₂₁₂Bi, ₁₃₁I, ₁₃₁In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The antibody can be conjugated to a “receptor” (such streptavidin) for utilisation in tumour pre-targeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Cancer chemotherapeutic agents that the antibody/antibody fragments or antibody derivatives can be associated with (either by conjugation or otherwise) include: alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Anti-metabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum co-ordination complexes such as cisplatin (cis-DDP) and carboplatin; anthracenedione such as mitoxantronei and anthracycline; substituted urea such as hydroxyurea; methyl hydrazine derivative such as procarbazine (N-methylhydrazine, M1H); and adrenocortical suppressant such as mitotane (o,p ′-DDD) and aminoglutethimide; taxol and analogues/derivatives; and hormone agonists/antagonists such as flutamide and tamoxifen.

### Example 9

### Stabilised Antibody—Nuclease Conjugate

Plasmid Construction

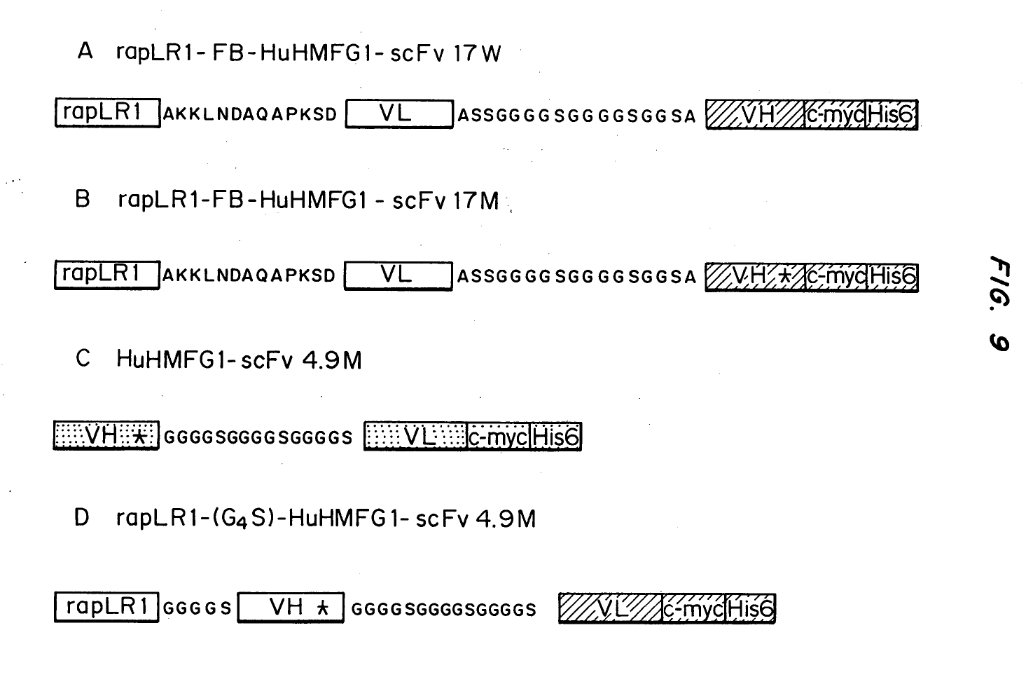

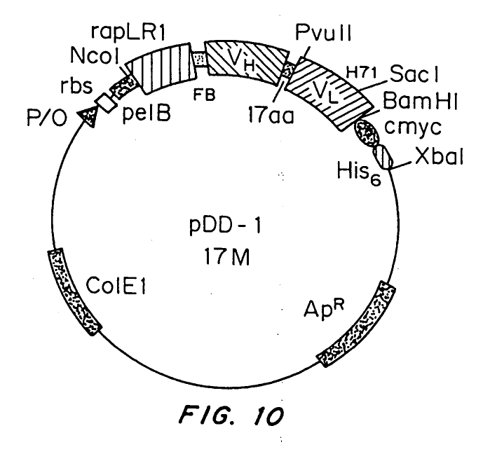

RapLR1-FB-huHMFG1-scFv 17M (FIG. 9B) was constructed by ligation of the PVUII-SacI fragment of scFv 4.9M (FIG. 9C) with the V_{H}-R71A mutation into the PVUII-SacI restricted pDD-1 vector containing rapLR1-FB-HuHMFG1-scFv 17W (FIG. 9A, FIG. 10). The FB spacer comprises 13 amino acids composed of residues 48-60 of fragment B (FB) of staphylococcal protein A (Tai et al. (1990) Biochemistry, 29, 8024-8030).

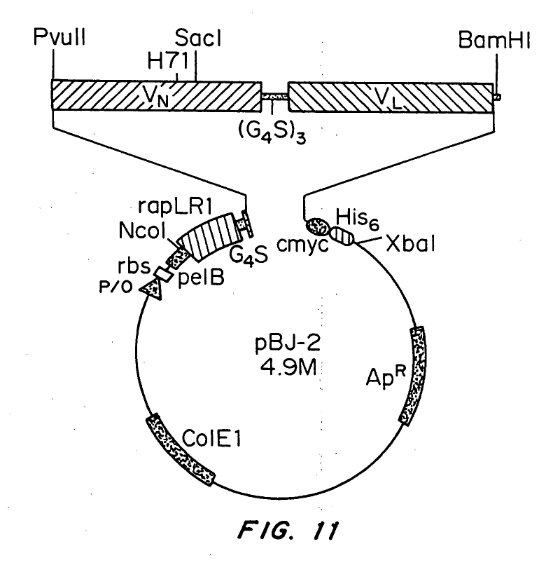

The nature of the N-terminus has been shown to be important to amphibian RNases (reviewed in Rybak & Newton (1999) Exp. Cell Res., 253, 325-335). Recombinant amphibian RNases expressed without a free N-terminal amino acid express neither catalytic nor cytotoxic activity. RapLR1-(G₄S)-huHMFG1-scFv 4.9M was obtained by ligating the PVUII-BamHI fragment of scFv 4.9M into the PVUII-BamHI restricted pBJ-2 vector containing the rapLR1 gene and a GGGGS spacer (FIG. 9D, FIG. 11) (SEQ ID NO: 7). The correct sizes and sequences of inserts were verified by analytical restriction digest and DNA sequencing, respectively.

Protein Expression and Purification

E. coli TG1 cells transformed with the plasmid encoding RapLR1-(G₄S)-huHMFG1-scFv 4.9M were grown at 37° C. overnight in 50 ml 2YT media containing 200 μg/ml ampicillin and 2% glucose. The cells were diluted into 4 L 2YT media containing 200 μg/ml ampicillin and grown at 37° C. until an OD₆₀₀ of 1.0 was reached. The media was adjusted to contain 1.0 mM IPTG. Induction was performed at 26° C. for 16-18 hrs.

The bacteria were centrifuged at 10,000×g for 45 min at 4° C. The fusion protein was isolated from inclusion bodies, denatured, renatured, and dialysed as described (Newton et al. (1996) Biochemistry, 35, 545-553). The extensively dialysed protein solution was applied to a 40 ml CM-Sephadex C-50 column (Pharmacia Biotech Inc., Piscataway, N.J.) equilibrated with 20 mM Tris-HCl, pH 7.5 containing 10% glycerol and eluted with equilibration buffer containing 1.0 M NaCl. The protein solution was diluted 10 fold with 50 mM NaOAc, pH 5.0, immediately applied to a 2 ml SP-Sepharose column (Pharmacia Biotech Inc., Piscataway, N.J.) and eluted with a 200 ml linear NaCl gradient (0.1-0.7 M) in 50 mM NaOAc, pH 5.0. RapLR1-FB-huHMFG1-sFv 17M was expressed in TG1 cells and purified as described in (Newton et al. (1996) Biochemistry, 35, 545-553).

Cytotoxicity Assay

MCF-7 cells (2500 in 0.1 ml) (ATCC # HTB-22) were placed in each well of a 96-well plate 24 hrs before treatment. On the day of treatment, test samples (10 μL) were added to the appropriate well, and the cells were incubated for 3 days at 37° C. in a humidified CO₂ incubator. To determine protein synthesis, the medium containing serum was replaced with serum- and leucine-free RPMI medium. [¹⁴C]Leucine (3.7 MBq in 10 μL) was added and the incubation continued for 2-4 h at 37° C. The cells were then harvested onto glass fibre filters using a cell harvester and the filters were washed with H₂O, dried in ethanol, and radioactivity was determined using a scintillation counter. Measurements were performed in triplicate and experiments performed at least twice.

The concentration of test sample which inhibits protein synthesis by 50% (IC₅₀) was determined from semi-logarithmic plots in which protein synthesis as a percentage of control (buffer-treated cells) was plotted versus test protein concentration.

Results

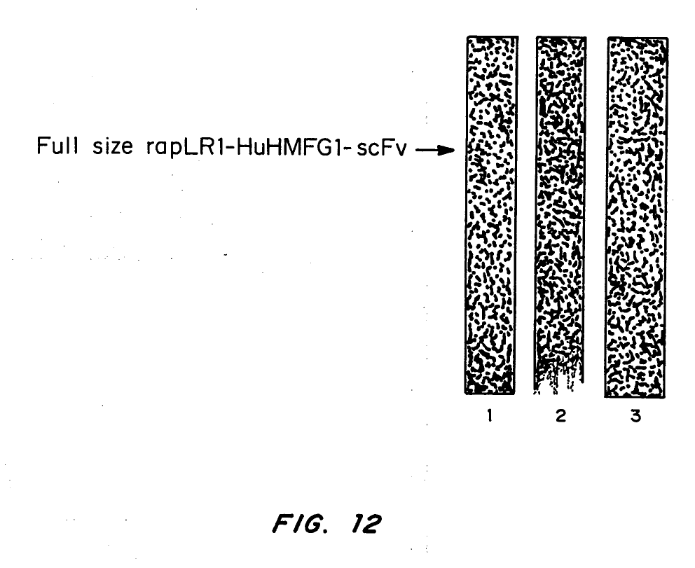

The initially constructed fusion protein rapLR1-FB-huHMFG1-scFv 17W (FIG. 9A) proved to be very unstable and difficult to purify to homogeneity (FIG. 12, lane 1).

To study the impact of the stabilising V_{H}-R71A mutation on the stability of the fusion protein rapLR1-FB-huHMFG1-scFv 17M was made (FIG. 9B, FIG. 10). As shown in FIG. 12, lane 2, the introduction of this single mutation did in fact allow for the purification of the fusion protein.

Analysis of this construct for enzymatic and binding activity showed that the fusion protein contained 41% the ribonuclease activity of rapLR1 and 27% the binding activity of the HMFG1 IgG antibody as determined by flow cytometry.

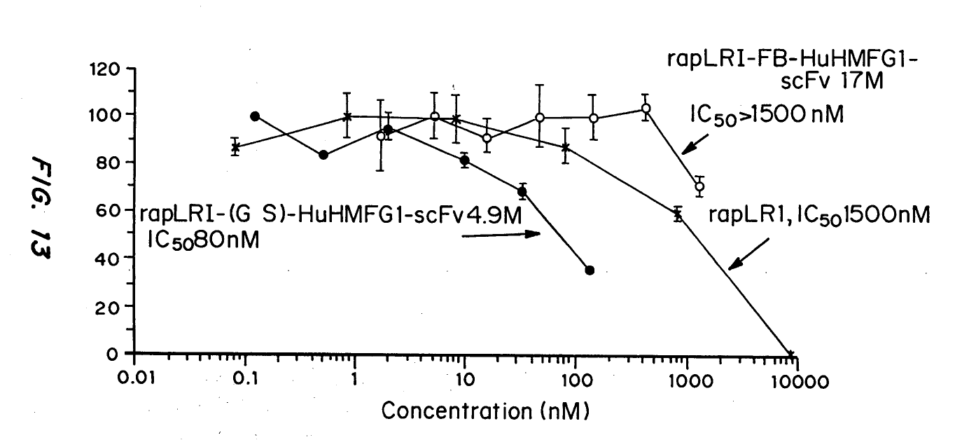

The cytotoxic effect of rapLR1-FB-huHMFG1-scFv 17M was assessed by measuring the [¹⁴C]leucine incorporation into newly synthesised proteins. As shown in FIG. 13, this protein was not able to inhibit protein synthesis in MCF7 cells (IC₅₀>1500 nM) suggesting a linker length of 17 amino acid residues not to be sufficient for maintaining stability upon incubation at 37° C. in serum.

A further fusion protein, rapLR1-(G₄S)-huHMFG1-scFv 4.9M (FIG. 9D, FIG. 11), was made which incorporates the following features: a) rapLR1 is attached to the amino terminus of a variable domain via the short GGGGS (SEQ ID NO: 7) spacer, thus allowing the amino terminus of rapLR1 to be unencumbered; b) the V_{H} domain contains the mutation responsible for the production of a more stable scFv with a higher affinity for the receptor; c) the orientation of the variable domains in the scFv is V_{H}-V_{L}; and d) the V_{H} and V_{L} domains are connected by a (GGGGS)₃ linker allowing for the constraint free pairing of the two variable domains. As shown in FIG. 12, lane 3, this fusion 10 protein could be purified to homogeneity.

Activity measurements of both components of the fusion protein demonstrated that 15% of the ribonuclease activity of rapLR1 and 20% the binding activity of the HMFG1 IgG was retained. In contrast to rapLR1-FB-huHMFG1-scFv 17M, however, this construct was active in killing MCF7 cells (IC₅₀ of 80 nM, FIG. 13) and exhibited approximately 18 fold stronger cytotoxic activity when compared with rapLR1 alone (IC₅₀ for native rapLR1, 1500 nM, FIG. 13).

### Example 10

### Pharmaceutical Formulations and Administration

A further aspect of the invention provides a pharmaceutical formulation comprising a compound according to the first aspect of the invention in admixture with a pharmaceutically or veterinarily acceptable adjuvant, diluent or carrier.

Preferably, the formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrastemally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for examples the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that he overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A modified antibody molecule which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target.

2. The modified antibody molecule of claim 1, wherein the site of modification is at one or more of the corners of the inner faces of the beta-sheets of the antibody molecule.

3. The modified antibody molecule of claim 1, wherein the site of the modification is the amino acid residue V_{H}71 in the amino acid sequence of FIG. 8 (SEQ ID NO: 1) or the corresponding residue in another antibody molecule.

4. The modified antibody molecule of claim 1, wherein the specific target is the MUC-1 gene product.

5. The modified antibody molecule of claim 1, wherein the modified amino acid possesses at least one physicochemical property different to the amino acid before modification.

6. The modified antibody molecule of claim 5, wherein the physicochemical property is at least one selected from group consisting of charge, hydrophobicity/hydrophilicity, size, structure, volume, polarity, and side chain characteristics.

7. The modified antibody molecule of claim 6, wherein the size of the amino acid is reduced by the modification.

8. The modified antibody molecule of claim 1, wherein the modification results in the amino acid, alanine.

9. The modified antibody molecule of claim 1, wherein the modification increases the buried surface area between motifs of the antigen binding site.

10. The modified antibody molecule of claim 1, wherein the modified antibody molecule has an antigen binding selectivity equivalent to gamma 1, kappa anti-HMFG monoclonal antibody.

11. The modified antibody molecule of claim 1, wherein the modified antibody molecule is a single chain antibody.

12. The modified antibody molecule of claim 11, wherein the single chain antibody is a single chain Fv.

13. The modified antibody molecule of claim 12, wherein the single chain antibody is a diabody.

14. The modified antibody molecule of claim 1, wherein the modified antibody molecule is humanized.

15. The modified antibody molecule of claim 14, wherein a humanized amino acid residue is modified to the murine amino acid.

16. The modified antibody molecule of claim 1, wherein the modified antibody molecule is associated with at least one other agent.

17. The modified antibody molecule of claim 16, wherein the agent is selected from the group consisting of drugs, toxins, radionuclides, nucleases, enzymes, cytokines and chemokines.

18. The modified antibody molecule of claim 16, wherein the agent is conjugated to the modified antibody molecule.

19. The modified antibody of claim 16, wherein the agent is a nuclease.

20. A nucleotide sequence encoding a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target.

21. The nucleotide sequence of claim 20, wherein the nucleotide sequence is that of FIG. 8 (SEQ ID NO: 2).

22. An expression vector containing a nucleotide sequence encoding a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target.

23. The expression vector of claim 22, wherein the nucleotide sequence is that of FIG. 8 (SEQ ID NO: 2).

24. A host cell producing a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target, resulting from the expression of the nucleotide sequence encoding the modified antibody molecule.

25. The host cell of claim 24, wherein the nucleotide sequence is that of FIG. (SEQ ID NO: 2).

26. A composition comprising a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target, and a pharmaceutically acceptable carrier, excipient and/or diluent.

27. A composition comprising a nucleotide sequence encoding a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target.

28. The composition of claim 26 further comprising at least one other agent.

29. The composition of claim 27 further comprising at least one other agent.

30. The composition of claim 28 wherein, the agent is selected from the group consisting of drugs, toxins, radionuclides, nucleases, enzymes, cytokines and chemokines.

31. The composition of claim 29 wherein, the agent is selected from the group consisting of drugs, toxins, radionuclides, nucleases, enzymes, cytokines and chemokines.

32. A method for the treatment and/or diagnosis and/or prevention of a disorder selected from the group consisting of cancer, inflammatory disorders, cardiovascular diseases, infectious diseases, autoimmune disorders, central nervous system disorders, nephritis, sepsis, haemoglobinuria, chemotherapy induced thrombocytopenia, and addiction comprising administering an effective amount of a modified antibody molecule, which selectively binds to a specific target, the antibody molecule being modified at, at least one amino acid residue that determines antigen binding selectivity and/or affinity, wherein the modified antibody molecule exhibits a greater stability than an unmodified parent antibody molecule, which selectively binds to that target.

33. The method of claim 32, wherein the disease is cancer.

34. The method of claim 33, wherein the cancer is selected from the group consisting of cancer of the breast, ovary, uterus, lung, B-cell non-Hodgkins lymphoma, multiple myeloma, acute myelogenous leukemia, chronic lymphocytic leukemia and hairy cell leukemia.

35. The nucleotide sequence of claim 22 wherein the sequence is in an expression phage.

36. The nucleotide sequence of claim 5 wherein the nucleotide sequence is operably linked to the nucleotide sequence encoding a phage surface protein.

37. The nucleotide sequence of claim 6 wherein the phage expresses and displays the product of the sequence.

38. A method of screening of antibodies, antibody fragments or antibody derivatives that are able to bind a target molecule comprising providing the phage of claim 35 and one or more target molecules.

39. The method of claim 38 wherein the target molecules is a MUC1 gene product.
